(19) United States
(12) Patent Application Publication
(10) Pub. No.: US 2016/0175608 A1
(43) Pub. Date: Jun. 23, 2016

(54) LIGHT EMITTING DEVICE AND METHOD OF USING THE SAME

(71) Applicant: Troy William Livingston, Buffalo Grove, IL (US)

(72) Inventor: Troy William Livingston, Buffalo Grove, IL (US)

(21) Appl. No.: 14/970,448

(22) Filed: Dec. 15, 2015

Related U.S. Application Data
(60) Provisional application No. 62/093,279, filed on Dec. 17, 2014.
(60) Provisional application No. 62/096,736, filed on Dec. 24, 2014.

(51) Int. Cl. A61N 5/06

(52) U.S. Cl. CPC A61N 5/0613; A61N 2005/064; A61N 2005/0651; A61N 2005/0654; A61N 2005/0661

(57) ABSTRACT

A light emitting device and method of using the same is provided. The light emitting device has a plurality of moveable panels forming a cabinet which can be altered to increase or decrease the amount of UV the patient using the device receives on various areas of the patient's body. The panels are connected by a plurality of hinges located between the plurality of panels. Each panel may have a plurality of UV lamps/diodes which provide the UV light to the patient for the light emitting session. A control panel located allows the patient (or operator) to control the UV output. The light emitting device also allows for the flexible interconnection of power and signal cables so as to reduce stress and prolong the lifespan of the power and cable lines used in the light emitting device.

### CROSS REFERENCE TO RELATED APPLICATIONS

The following application is based on U.S. provisional application Ser. No. 62/093,279 filed on Dec. 17, 2015 and Ser. No. 62/096,736 filed on Dec. 24, 2015 both currently co-pending, and claims the priority benefit of the '279 and the '736 U.S. applications; the entire contents of which are incorporated by reference.

### BACKGROUND OF THE INVENTION

A light emitting device and method of using the same is provided. The light emitting device has a plurality of moveable panels forming a cabinet which can be altered to increase or decrease the amount of UV light the patient using the device receives on various areas of the patient's body. The panels are connected by a plurality of hinges located between the plurality of panels. Each panel may have a plurality of UV lights, which provide the UV light to the patient for the therapy session. A control panel accessible to the patient (or operator) allows control of the UV output. The present light emitting device has a flexible interconnection of power and signal cables system so as to reduce stress and prolong the lifespan of the power and signal cables lines used in the light emitting device.

The interconnection of electrical components in moveable assemblies of medical products is often a problem for both electrical lines being able to withstand multiple flexing without breaking or shorting and in appearance where a flexible conduit is utilized making medical equipment appear industrial and not appealing to patients. The present light emitting device and method of using the same solves this problem by providing separate paths for power and data lines. Low flexibility power lines are given four feet of length to provide 180 degrees of flexibility so as to eliminate the stress on the electrical conductors. High flexibility data cables are provided a separate, shorter path between moveable members to eliminate the interference between power and data cables and to provide the minimum length possible for efficient transmission of electronic data. The present light emitting device and method of using the same provide a secure, low cost solution for flexible interconnection of power and signal cables with the advantage of hiding the conducting cable in a non-threatening, pleasing appearance device.

Conducting electrical power between moving assemblies is very difficult as the flexing motion quickly fractures copper electrical wires causing open electrical connections and dangerous breaks in the conductors as the strands are broken. The remaining strands must carry all the current and rapidly overheat from being overloaded. The only way to prevent this problem is by using high flex electrical wiring and severely limiting the number of flexes or the amount of flex required in the wire. Other alternatives exist such as electrical brushes and commutators or special spring conductors but they are extremely expensive.

The present light emitting device and method of using the same solves this electrical conduit problem by creating a longer pathway for the electrical conductors to reduce the stress on these conductors to an acceptable level for using low cost power and data conductors.

The present light emitting device and method of using the same utilize a PVC cable wire which is: highly flexible, and has fine bare copper strands with VDE-0295 Class 5, IEC 60228 C1-5. The method spreads the wire 180° of flexure over 4 feet and therein reduces the strain on the wire to 3.75° degrees per inch of twist in the power cord. This low level of twist per inch enables the commercially available electrical conductors using the present light emitting device to meet the 100,000 flexure test required for safety regulatory testing of products per harmonized international standards.

Cost of electrical products is a serious issue. Four feet of the high flex wire and a flexible conductor assembly provide a minimum cost alternative. Therefore utilizing the multi tube and sleeve design in the present light emitting device, low cost wire may be safely used and therefore the need to add a complex and expensive flexible electrical assembly is eliminated.

Structural integrity and electrical capabilities of the rotating hinge created by the present light emitting device is assured by the multi-tube methodology shown by the preferred embodiment of the present light emitting device and method of using the same.

Attempts have been made to provide light emitting device and method of using the same. For example, U.S. Pat. No. 8,518,094 to Wang discloses a guided light emitting apparatus for treating soft tissue injury, chronic pain, and promoting wound healing for both human and animal targets. The light emitting apparatus comprises sensors for monitoring the intensity, position, and movement of the therapeutic light beam over the treatment area. The delivered light energy dosage is determined accordingly based on these parameters. The light emitting apparatus further comprises a projector device for projecting markers on top of the treatment area. The markers represent the values of the delivered light energy dosage for assisting the practitioner or clinician in precisely controlling the light emitting procedure.

Further, U.S. Pat. No. 6,984,228 to Anderson discloses methods and systems for treating inflammatory, proliferative skin disorders, such as psoriasis, with ultraviolet light emitting. The methods and systems use optical techniques to scan a patient's skin, designate areas of affected skin, and selectively deliver high doses of phototherapeutic ultraviolet radiation to the designated areas. To insure that only affected areas of skin affected are designated for the high doses of UV radiation, the methods and systems use one or more optical diagnostics that relate to independent physiological features of affected skin. Still further, U.S. Pat. No. D517,699 to Lansohn and U.S. Pat. No. D272,093 to Fish disclose light emitting devices.

However, these patents fail to provide a light emitting device and a method of using the same as described in the present application. A need, therefore, exists for an improved light emitting device that has the features of the present invention.

### SUMMARY OF THE INVENTION

A light emitting device and method of using the same is provided. The light emitting device has a plurality of moveable panels forming a cabinet which can be altered to increase or decrease the amount of UV the patient using the device receives on various areas of the patient's body. The panels are connected by a plurality of hinges located between the plurality of panels. Each panel may have a plurality of UV lights which provide the UV light to the patient for the light emitting session. A control panel accessible to the patient (or operator) allows control of the UV output. The light emitting device also allows for the flexible interconnection of power and signal cables so as to reduce stress and prolong the lifespan of the power and cable lines used in the light emitting device.

An advantage of the present light emitting device and method of using the same is that the present light emitting device and method allows for reduced stress of movable power and data lines within medical equipment.

Yet another advantage of the present light emitting device and method of using the same is that the present light emitting device and method of using the same extends the life span of movable power and data lines within medical equipment.

Still another advantage of the present light emitting device and method of using the same is that the present light emitting device and method of using the same allows for accurate delivery of UV light to specific areas of the patient as a result of a plurality of panels of the light emitting device being able to be configured in various orientations.

And another advantage of the present light emitting device and method of using the same is that the present light emitting device and method of using the same is that the present light emitting device has a UV eye shield which may be easily altered depending on the height of a patient being treated.

For a more complete understanding of the above listed features and advantages of the light emitting device reference should be made to the following detailed description of the preferred embodiments and to the accompanying drawings. Further, additional features and advantages of the present invention are described in, and will be apparent from, the detailed description of the preferred embodiments and from the drawings.

### BRIEF DESCRIPTION OF THE DRAWINGS

FIG. 1 illustrates a front perspective view of the light emitting device in a usable First Orientation.

FIG. 2 illustrates a top view of the light emitting device in the usable First Orientation wherein a patient is within the interior of the light emitting device.

FIG. 3 illustrates a top view of an alternative Second Orientation of the light emitting device.

FIG. 4 illustrates a front view of the connection hinge between two panels of the light emitting device.

FIG. 5 illustrates a cross section of the interior of the connection hinge between two panels of the light emitting device wherein the power and data lines are visible.

FIG. 6 illustrates a side view of the UV eye shield of the present device wherein the UV eye shield is in the folded half down orientation.

FIG. 7 illustrates a side view of the UV eye shield of the present device wherein the UV eye shield is in the full down orientation.

FIG. 8 illustrates a side view of the UV eye shield of the present device wherein the UV eye shield is in the up orientation.

### DETAILED DESCRIPTION OF THE PREFERRED EMBODIMENTS

A light emitting device and method of using the same is provided. The light emitting device has a plurality of moveable panels forming a cabinet which can be altered to increase or decrease the amount of UV the patient using the device receives on various areas of the patient's body. The panels are connected by a plurality of hinges located between the plurality of panels. Each panel may have a plurality of UV lights, which provide the UV light to the patient for the light emitting session. A control panel accessible to the patient (or operator) allows control of the UV output. The light emitting device also allows for the flexible interconnection of power and signal cables so as to reduce stress and prolong the lifespan of the power and cable lines used in the light emitting device.

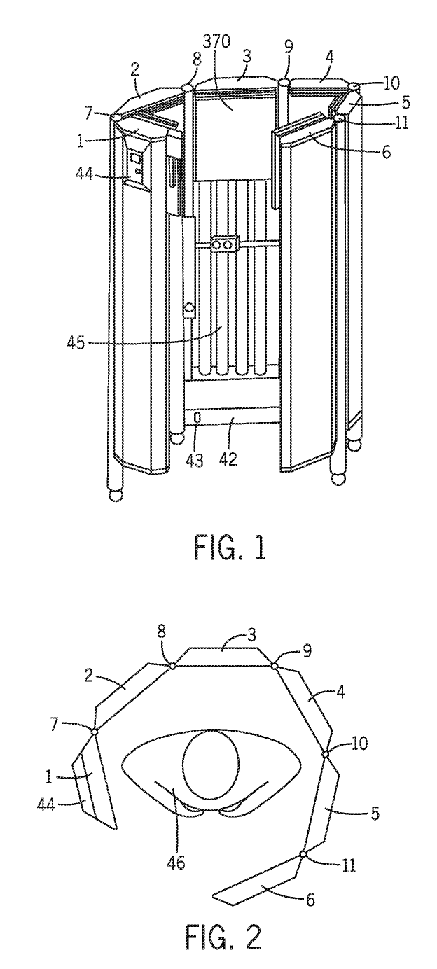

Referring now to the Figure FIG. 1 is an isometric view of the front most view of the present light emitting device showing the semicircular opening operated by lamp/diode panels 1, 2, 3, 4, 5, 6 supported by the present light emitting device hinge tubes 7, 8, 9, 10, 11.

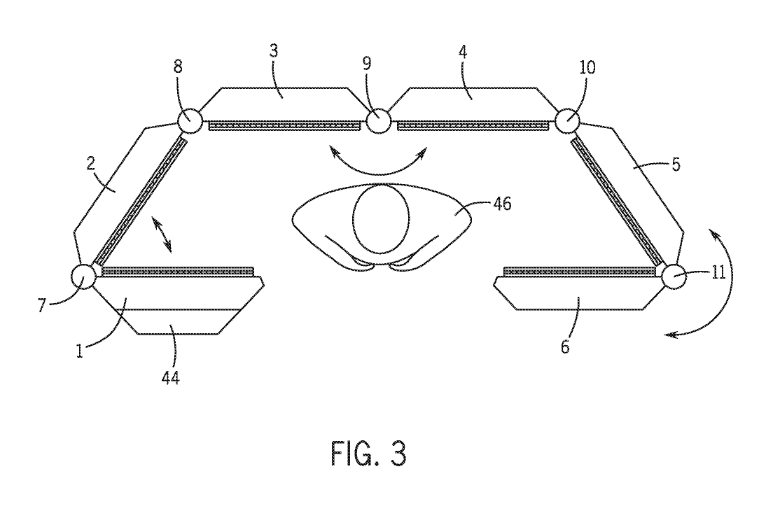

FIG. 2 is a top most view of the semicircular opening created by lamp/diode panels 1, 2, 3, 4, 5, 6 with hinge tubes 7, 8, 9, 10, 11 respectively connecting lamp/diode panels 1,2, 3, 4, 5, 6 and providing the electrical interconnection between the respective lamp/diode panels and the hinging flexibility as shown in FIG. 3.

FIG. 3 shows a top view of lamp/diode panels 1, 2, 3, 4, 5, 6 in a second embodiment. Hinge tubes 7, 8, 9, 10, 11 provided electrical connection to the respective adjoining lamp/diode panels and the hinging capability to safely and securely repeatedly fold lamp/diode panels 1, 2, 3, 4, 5, 6 from FIG. 2 to the FIG. 3 and return configurations.

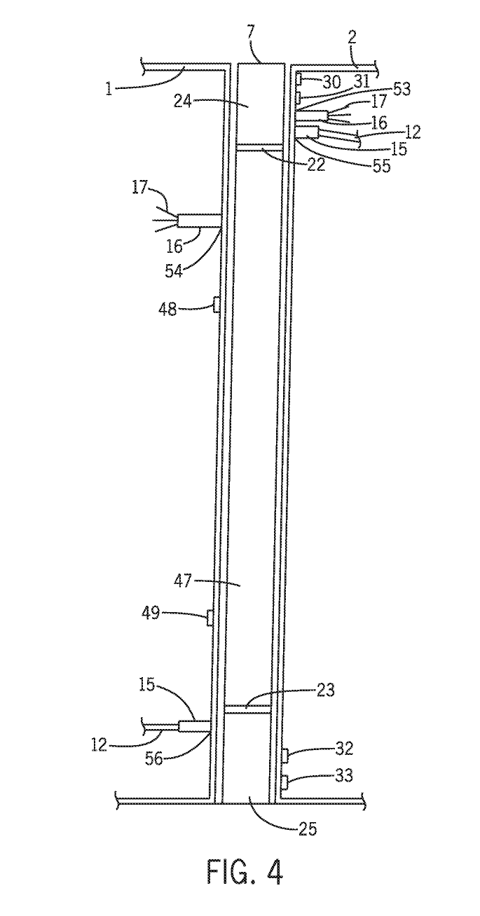

FIG. 4 is a cut away of the present light emitting device hinge tube 7 in its preferred embodiment showing high voltage power cord 12 and data line 16 entering top hinge tube 24 from lamp/diode panel 2 at top right most position 53 and exiting at bottom left most position 54 of lamp/diode panel 1. Data lines 17 and 18 are shown entering both insulating tube 16 at top right most position of top hinge tube 24 and exiting at left top most position of center hinge tube 47 to provide data connection between lamp/diode panels 1 and 2 respectively.

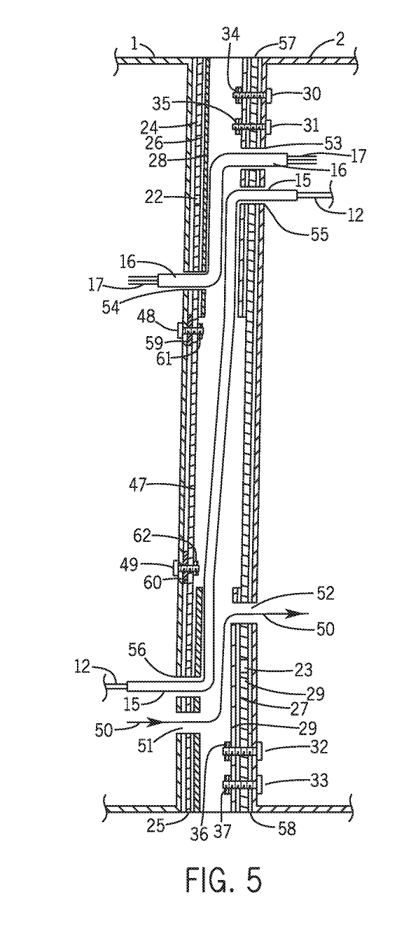

FIG. 5 is a cutaway detail view of the present light emitting device showing the high voltage power line supported by insulating tube 15 entering power port 55 within panel 2 and exiting power port 56 within panel 1. This conduit path provides effective power transmission between panels 1 and 2. Data transmission lines 17 enter data port 53 in panel 2 and exit data port 54 in panel 1. This provides a conduit electrical path for data transmission between panels 1 and 2 respectively: Structural integrity of supporting lamp/diode panel 1 and lamp/diode panel 2 in their O to 180 degree of flexing relative to each other is provided by hinge tube 7 comprised of top hinge tube 24, center hinge tube 47, bottom hinge tube 25 and interval components further defined in the detailed description below.

FIG. 1 shows the present light emitting device, hinge tube 7,8,9,10,11 connecting lamp/diode panels 1,2,3,4,5. Hinge tubes 7,8,9, 10,11 allow the respective lamp/diode panels to flex from O to 180 degrees to each adjoining lamp/diode panel. It is essential for the comfort of the patient 46 in the medical device that the patient 46 is not negatively affected by the device appearance to minimize patient 46 stress in the light therapy process. Therefore, the device must not have protrusions or appear industrial to provide a level of comfort to the patient 46. Lamp/diode panels 1, 2, 3, 4, 5, 6 are interconnected by hinge tubes 7,8,9, 10,11 to provide 180 degrees at flexibility between respective panels.

FIG. 2 shows a top view of the preferred embodiment of the present light emitting device with patient 46 centered within the generally circular opening 39 formed by lamp/diode panels 1,2.3,4,5,6 and hinge tubes 7,8,9.10, 11. Electrical power and data is input through input panel 42 and interconnected to lamp/diode panels 1,2,4,5,6 through hinge tubes 7,8,9,10,11 respectively.

FIG. 3 is a top view of the second orientation wherein the panels 1,2,3,4,5,6 and hinge tubes 7,8,9, 10,11 are being utilized in the safe transmission of electrical power and data to the respective adjoining lamp/diode panels during the movement between the first orientation (FIG. 2) and the second orientation (FIG. 3). FIG. 3 illustrates that the panels 3,4 may be rotated approximately one hundred and eighty degrees with respect to one another so that the panels 3, 4 are generally on the same plane. Further, FIG. 3 illustrates that the panels 5, 6 may also electively be rotated at the hinge tubes 7, 8, 9, 10, 11 such that the angle with respect to each other is not one hundred and eighty degrees (in the case of panel 5 and 6 in FIG. 3 the panels 5 and 6 are approximately at a forty-five degree angle with respect to one another. It should be understood that any two adjacent panels may be rotated at virtually any angle so as to allow the operator to create a light emitting machine having various interior configurations which therein increase or decrease exposure of the UV light at various locations on the body of the patient 46.

FIG. 4 is a detail view of the outside surface of the present light emitting device 7. Lamp/diode panel 1 is illustrated near the left side of the top hinge tube 24, center hinge tube, 47 and bottom hinge tube, 25 is secured to center hinge tube 47 by bolts 48,49. This allows center hinge tube 47 to support top hinge tube 24 and bottom hinge tube 25 are securely fastened near the adjoining right portion of the lamp/diode panel 2 by bolts 30,31 and 32,33 respectively. Power line 12 is protected by insulation, 15 and enters top hinge tube, 24 at entry port 55 and exits at bottom hinge tube 25 at entry port 56. The 4 foot distance between entry port 55 and entry port 56 allows the 180 degree maximum rotation flexibility of lamp/diode panel 1 and lamp/diode panel 2. Relative to each other, this 4′ span provides adequate strain relief in the 180 degree of rotation to not exceed this twisting specification of 11OV power cable such as PVC cable wire which is highly flexible, and has fine bare copper strands with VDE-0295 Class 5, IEC 60228 C1-5 (for example, flex-52 3G2, 5 by Kasel Tee). Data line 17 in data insulation 16 entering top tube 24 at entry port 53 and exiting at data port 54 is provided this shorter path between data entry port 55 and data entry port 54 to minimize the electrical interference between power line 12 and data lines 17. Spacers 22, 23 provide bearing surfaces to reduce the rotating friction between top hinge tube 24, center hinge tube 47 and bottom hinge tube 25.

FIG. 5 is a detailed side cross-section view of the preferred-embodiment of the present light emitting device. Left and right lamp/diode panels 1 and 2 are shown as a 2 panel hinge tube set in this present light emitting device preferred embodiment. Multiple sets of this preferred embodiment do not diminish or limit the present light emitting device and protection of intellectual property. Lamp/diode panel 1 is securely fastened to the center hinge tube 4 by bolts 48, 49, through spacers 59, 60 to nuts 61, 62 respectively. This secure mounting enables data line 17 to exit center hinge tube 47 and into panel 1 with data insulation 16 protecting data lines 17 transition through entry port 53,54 respectively. Top hinge tube 24 and bottom hinge tube 25 are separated near their top most and near their bottom most ends by spacer 22,23 respectively for reduction of friction between top hinge tube 25, center hinge tube 47 and bottom tube 25. Top hinge tube 24 is securely fastened to lamp/diode panel 2 at its further right most side by bolts 30,31 to nuts 34,35 respectively. Outer most surface of top inside tube 28 is contained by the innermost surface of top sleeve bearing 26. Outermost surface of top sleep bearing 26 is in intimate contact and contained by the innermost surface of top hinge tube 24. Top hinge tube 24, top sleeve bearing 26 and top inside tube 28 combine to form the support of the top most end of hinge tube 7 FIG. 1. Outermost surface of bottom inside tube 29 is in contact with the inside most surface of bottom sleeve bearing 27 outermost surface of bottom sleeve bearing 27 is in contact with innermost surface of bottom hinge tube 25. This combination of bottom inside tube 29, bottom sleeve bearing 27, and bottom hinge tube 25 form the support of the lower most end of center hinge tube 47. Therefore panel 1 and panel 2 being securely fastened to center hingetube 47 by bolts 48,49 and top hinge tube 24 and bottom hinge tube 25 being securely fastened to panel 1 and panel 2 by bolts 30,31,32,36 respectively are able to pivot from 0 to 180 degrees to each other in a smooth and safe motion. Alternate data path 50 is provided with data port 52 and data port 51 respectively as an additional conduit path for additional data lines as shown in data line 17 entry port 53 and data line 17 entry port 54. A smooth, quiet, long life, 0 to 180 Degree hinge with low stress power and data line conduit is provided with the present light emitting device allowing medical equipment to meet esthetic and functional requirements for patient 46 comfort and safety.

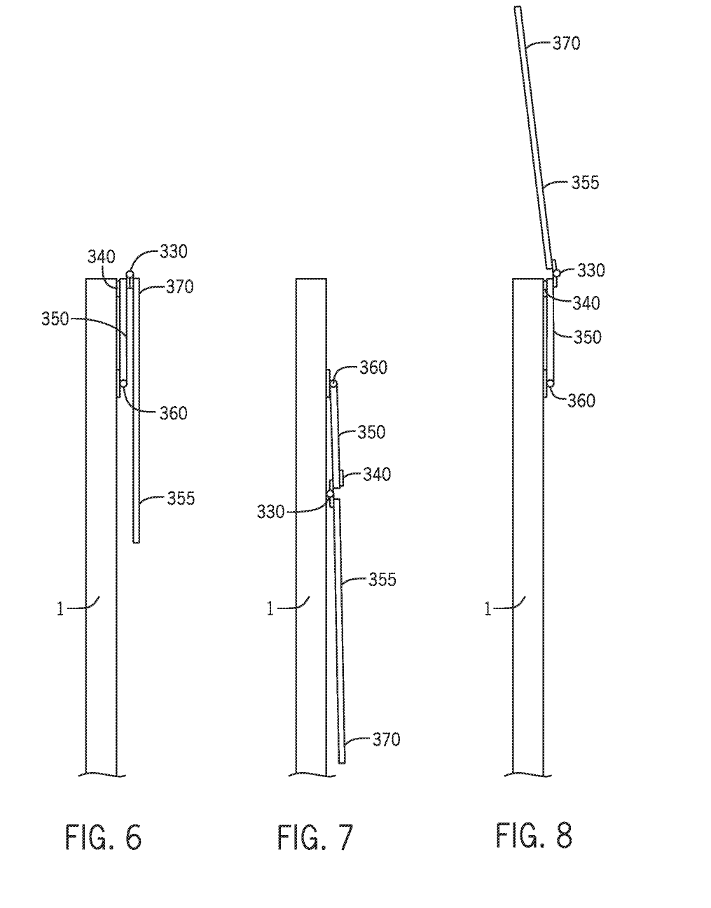

Referring now to FIGS. 6-8, in an embodiment, the present light emitting device may utilize at least one UV eye shield 370 secured to at least one panel 1. The UV shields 370 may provide eye protection for a patient 46 from direct eye exposure from the UV light source located in each lamp/diode panel 1. Eye protection is essential to the safe operation of the present light emitting device. Eye protection glasses may be required for patient 46 as an additional safety factor. Preferably, the UV shields 370 are configured to meet the requirements of a nominal height patient 46 from, for example, four feet to seven feet.

The UV eye shield 370 may have a first unit 350, a second unit 355, a hinge 330 and a magnet 340. Preferably, the first unit 350 is smaller than the second unit 355. The first unit 350 may be separated from the second unit 355 by the hinge 330 so that the first unit 350 may rotate approximately one hundred and eighty degrees with respect to the second unit 355. A second, and permanent and stationary, hinge 360 may connect the UV eye shield 370 physically to the panel 1 therein allowing the UV eye shield 370 to pivot at the first unit 350. In an embodiment, the first unit 350 and the second unit 355 may be opaque so as to block light and further may be generally square or rectangular in shape.

FIG. 6 illustrates the second unit 355 folded in the half-down orientation. When the UV eye shield 370 is in the half-down orientation, the UV light exposure to the patient 46 from the portion of the panel 1 directly behind the UV eye shield 370 is prevented from reaching the patient 46. To keep the UV eye shield 370 in the half-down orientation, the magnet 340 of the first unit 350 is brought into contact with a metallic surface on the panel 1. In the half-down orientation, substantially all of the first unit 350 is located between the second unit 355 and the panel 1 such that the first unit 350 is concealed behind the second unit 355 during use (when viewed from within the interior of the light emitting machine).

In order to increase the area the UV eye shield 370 prevents the UV light from reaching the patient 46, the UV eye shield 370 may be moved into the fully downward orientation (FIG. 7). In this orientation, a greater percentage of the panel 1 behind the UV eye shield 370 is covered and therein less UV light reaches the patient 46. The fully downward orientation of FIG. 7 is ideal for shorter patients 46. Further, in this full downward orientation, the first unit 350 and second unit 355 are approximately one hundred and eighty degrees with respect to each other and wherein the first unit 350 is located above the second unit 355 with respect to the ground.

Finally, for taller patients 46 the UV eye shield 370 may be moved into the upward orientation of FIG. 8. In this orientation, the second unit 355 is rotated above the first unit 350 and the magnet 340 is therein secured to the panel 1. The second unit 355 may be moved over the direct vertical axis of the first unit 350 such that gravity pulls the second unit 355 and keeps the second unit 355 in the upward orientation. In this orientation, there is maximum exposure to the UV light source in the panel 1. Further, in this upward downward orientation, the first unit 350 and second unit 355 are approximately one hundred and eighty degrees with respect to each other and wherein the second unit 355 is located above the first unit 350 with respect to the ground.

Although embodiments of the present invention are shown and described therein, it should be understood that various changes and modifications to the presently preferred embodiments will be apparent to those skilled in the art. Such changes and modifications may be made without departing from the spirit and scope of the present invention and without diminishing its attendant advantages. It is, therefore, intended that such changes and modifications be covered by the appended claims.

I claim:

1) A light emitting device comprising:
a housing having a plurality of panels wherein the plurality of panels each have a top, a bottom, a front, a back, a first side and a second side;
a UV light lamp/diode located on the front of or within an interior of at least two of the plurality of panels;
an elongated column located between the first side of one of the plurality of panels and the second side of an adjacent of the plurality of panels wherein the elongated column has a length and wherein the elongated column has a generally hollow interior;
a hinge mechanism connected to the elongated column wherein the hinge mechanism allows the first panel to rotate up to one hundred and eighty degrees with respect to the second panel;
an electrical power line having a first end, a second end and a middle section wherein the middle section of the electrical power line is located within the generally hollow interior of the elongated column;
a data line having a first end, a second end and a middle section wherein the middle section of the data line is located within the generally hollow interior of the elongated column;
wherein one of the plurality of panels may be rotated up to one hundred and eighty degrees with respect to a second of the plurality of panels at the elongated column; and
wherein the plurality of panels are collectively capable of forming a plurality of various different housing configurations and wherein the housing configurations each have an opening extending into an interior of the housing configuration and wherein a patient is capable of being located in the interior of each of the housing configurations and wherein the patient receives a light treatment from a UV light lamp/diode while in the interior of the housing configuration.

2) The light emitting device of claim 1 further comprising:
a control panel electrically connected to the UV light light source wherein the control panel allows an operator to alter the amount of light emitted from the UV light source.

3) The light emitting device of claim 1 wherein the power line is made from a high flex wire, PVC cable wire.

4) The light emitting device of claim 1 wherein the power line and data line rotate between three and four degrees per inch twist when one of the plurality of panels rotates with respect to the elongated column.

5) The light emitting device of claim 1 wherein an angle of a first panel of the housing to the second panel of the housing is different than an angle of the second panel to a third panel of the housing.

6) The light emitting device of claim 1 further comprising:
a first opening and a second opening located on the first side of one of the plurality of panels wherein the first opening and the second opening extend into an interior of one of the plurality of panels and wherein the first opening and the second opening are capable of receiving a portion of the electrical power wire line or data line.

7) The light emitting device of claim 1 further comprising:
a first opening and a second opening located on a side of the elongated column wherein the first opening and second opening are capable of receiving a portion of the electrical power wire or data line.

8) The light emitting device of claim 1 further comprising:
an opaque UV eye shield located and secured to the front of one of the plurality of panels of the housing wherein the opaque UV eye shield has a first unit, a second unit and a hinge wherein the first unit rotates with respect to the second unit at the hinge.

9) The light emitting device of claim 8 further comprising:
a magnet permanently secured to a front of the first unit of the opaque UV eye shield wherein the magnet is correspondingly and temporarily secured to a metallic portion of one of the plurality of panels so as to keep the first unit secured to one of the plurality of panels.

10) The light emitting device of claim 8 wherein the opaque UV eye shield moves from a first orientation wherein the first unit and second unit are approximately one hundred and eighty degrees with respect to each other to a second orientation wherein the first unit is located between the second unit and one of the plurality of panels and wherein the first unit is therein concealed by the second unit.

11) The light emitting device of claim 1 wherein the UV light source allows the patient to produce vitamin D by supply a predetermined amount of UV light from the UV light source.

12) 1) A light emitting device comprising:
a housing having a plurality of panels wherein the plurality of panels each have a top, a bottom, a front, a back, a first side and a second side;
a UV light source located on the front of or within an interior of at least two of the plurality of panels;
an elongated column located between the first side of one of the plurality of panels and the second side of an adjacent of the plurality of panels wherein the elongated column has a length and wherein the elongated column has a generally hollow interior;
a hinge mechanism connected to the elongated column wherein the hinge mechanism allows the first panel to rotate up to one hundred and eighty degrees with respect to the second panel;
an electrical power line having a first end, a second end and a middle section wherein the middle section of the electrical power line is located within the generally hollow interior of the elongated column;
a data line having a first end, a second end and a middle section wherein the middle section of the data line is located within the generally hollow interior of the elongated column; and
an opaque UV eye shield located and secured to the front of one of the plurality of panels of the housing wherein the opaque UV eye shield has a first unit, a second unit and a hinge wherein the first unit rotates with respect to the second unit at the hinge.
